(19) United States
(12) Patent Application Publication
(10) Pub. No.: US 2005/0096518 A1
(43) Pub. Date: May 5, 2005

(54) Biological test system

(76) Inventor: Yu-Hong Chang, San-Chung City (TW)

Correspondence Address: Troxell Law Office PLLC, 5205 Leesburg Pike, Suite 1404, Falls Church, VA, 22041, US

(21) Appl. No.: 10/697,324

(22) Filed: Oct. 31, 2003

(51) Int. Cl. A61B 5/05

(52) U.S. Cl. 600/345; 435/4

(57) ABSTRACT

The invention discloses a new biological test system including a biological monitor which can transmit the processed data, analog or digital signals, via a communication port, whether USB, serial or parallel, and a communication media such as telephone, computer or PDA, to the internet server for data analysis and storage. The computer or PDA may download the software for data analysis on line from the web-station without transmitting the data to the internet server.

### BACKGROUND OF THE INVENTION

(a) Field of the Invention

The present invention generally relates to a biological test system and, more particularly, to the portable biological measurement of blood glucose, uric acid and cholesterol and their date management processes through the improvement of current biosensor monitor, incorporating the latest technology of wireless communication, internet and electronics.

(b) Description of the Prior Art

All the currently available biosensor monitors of blood glucose, uric acid or cholesterol, etc, used the design and practices which have not much change over the last 20 years, with no input of the latest development of internet, wireless communication, and electronics. Thus, the users of current biosensor monitor are as inconvenient as they were 20 years ago. The users enjoy nothing of the latest development of internet and wireless communication, and B to C (Business to Customer) service is nearly impossible.

### SUMMARY OF THE INVENTION

It is therefore the objective of the present invention to design a biological test system incorporating the latest development of internet service, wireless communication and electronics, to make data acquisition, storage and processing more efficient and less tedious for the users. Also, the manufacturers of monitors will have better scope of reach to the customers. Monitor users, as well as the medical professionals, will be able to access to the processed test data on the internet serve via computer with the authorized account number and password, so as to have better medical care and service. To achieve these objectives, a monitor equipped with the communication port such as USB, serial or parallel port, will be used to transmit the biological test data via wireless communication, such as a mobile phone, or via a computer, to the internet server, which will further process the acquired data into the format and style specially designed for medical professionals or the individual users. Furthermore, this biosensor monitor should be equipped with an integrated circuit board and a CPU, just like the conventional monitors, to process the response of the electrochemical reaction occurred on the test strip as the blood is applied, and to display such results on the LCD screen of the monitor, and further to transmit the signals via a mobile phone or linked computer to an internet server for more advanced data processing, which is accessible to the authorized personnel. With this system, the monitor manufacturer will be in precise control of the health condition of every individual and to provide proper medical advice and care, a good B to C (Business to Customer) service.

### BRIEF DESCRIPTION THE PREFERRED EMBODIMENT

This new biological test system of the present invention may be more readily understood by one skilled in the art with reference to the following detailed description taken in conjunction with the accompanying drawings wherein like elements are designated by identical reference numbers throughout the several views and, in which:

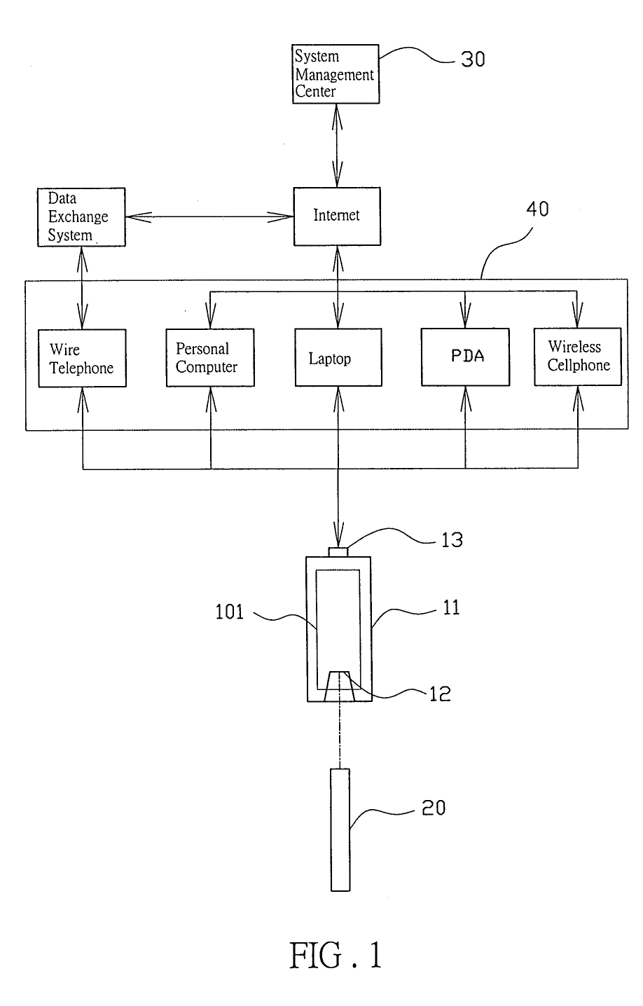

FIG. 1 is the operational flow diagram of the biological test system of the present invention.

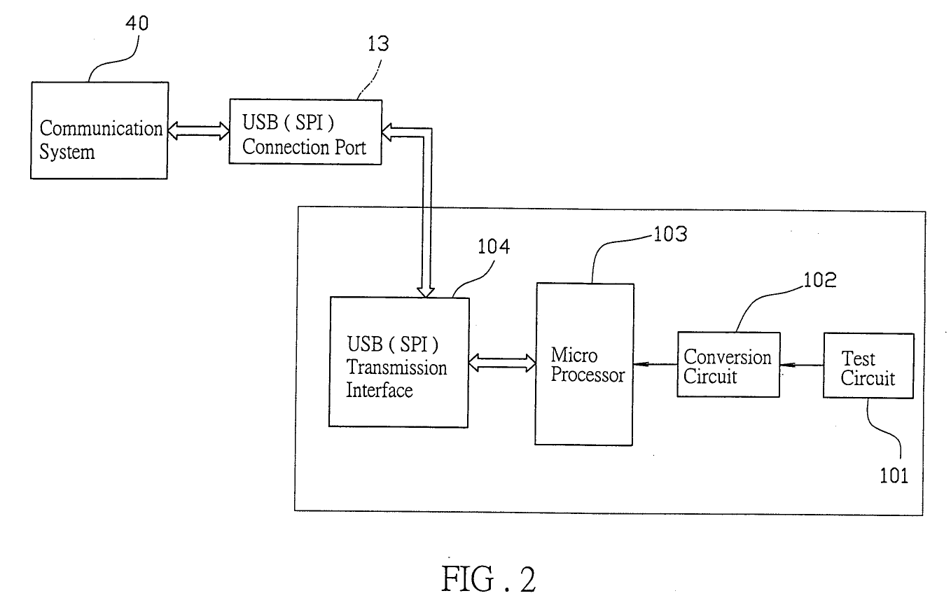

FIG. 2 illustrates the electronic block diagram of the monitor of the present invention.

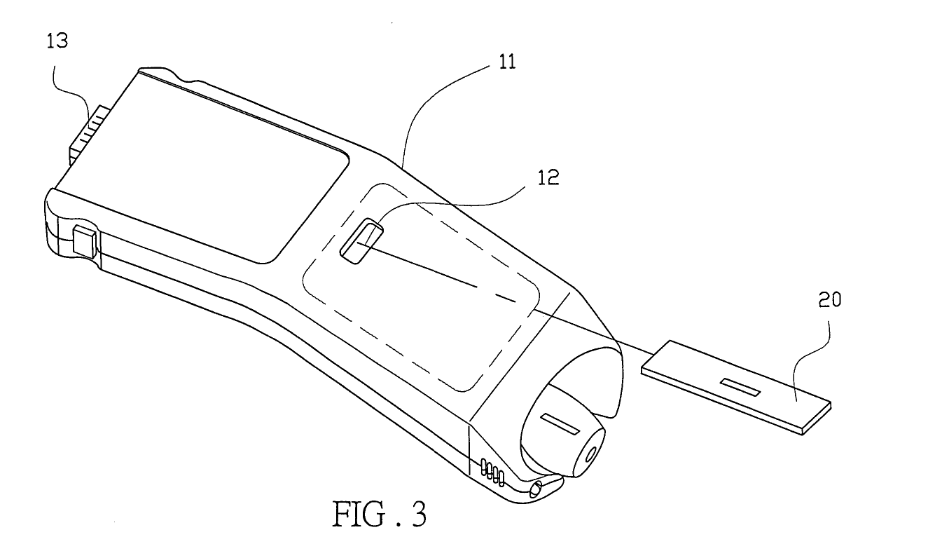

FIG. 3 is the elevational view of the monitor of the present invention.

### DETAILED DESCRIPTION OF THE PREFERRED EMBODIMENT

FIG. 1 is the operational flow diagram of the biological test system of the present invention, wherein a test strip with reagent 20 is inserted into the strip receiver 12 of the biological monitor 11, in which there is a integrated circuit board and a CPU 101 to process the signals from the electrochemical reaction of the test strip 20 when the blood is applied out the reagent of the test strip 20. Such processed signals can be transmitted through the communication media 40, which includes regular telephone at home or at offices, mobile phone, personal desktop computer (PC), notebook computer, and PDA (Portable Data Analyzer), which then further transmits such processed data through internet to the server 30 for more advanced data acquisition, storage and analysis. FIG. 2 illustrates the electronic block diagram of the monitor of the invention, wherein a measurement circuit 101 picks up the electrochemical response of the test strip 20 which will be processed by the analog to digital conversion circuit 102 and the microprocessor 103. The analyzed data will be transmitted through the communication interface 104, whether USB, serial or parallel, to the communication port 13 which links to the communication media 40, such as telephone, mobile phone, PDA or computer. Such monitor can be best viewed from the schematic drawing of FIG. 3.

It is to be understood that the drawings are designed for purposes of illustration only, and are not intended for use as a definition of the limits and scope of the invention disclosed.

1-2. (canceled)

3. A biological test system comprising:
a) a communications media; and
b) a biological monitor connected to the communications media and having:
i) a strip receiver with a test strip;
ii) a test circuit connected to the strip receiver;
iii) an analog to digital conversion circuit connected to the test circuit;
iv) a microprocessor connected to the analog to digital conversion circuit;
v) a transmission interface connected to the microprocessor; and
vi) a communications port connected to the transmission interface, wherein the test circuit measuring an electrochemical monitor response of a reaction on the test strip and transmitting test results to the conversion circuit and the micro processor for processing, and the transmission interface receiving analyzed data from the conversion circuit and the micro processor and transmitting the analyzed data through the communications port to the communications media.

4. The biological test system according to claim 3, wherein the communications port is selected from a group of ports consisting of USB, serial and parallel.

5. The biological test system according to claim 3, further comprising a system management center connected to the communications media through an internet connection, the communications media sending the test results to the system management center for analysis and storage.

6. The biological test system according to claim 3, wherein the communications media has an analyzing software, and analyzing and storing the test results.

7. The biological test system according to claim 3, wherein the communications media is selected from a group of consisting of a telephone, a personal computer, a laptop computer, a personnel data assistant, and a wireless cellular telephone.
